(19) United States
(12) Patent Application Publication
(10) Pub. No.: US 2006/0064077 A1
(43) Pub. Date: Mar. 23, 2006

(54) Method for correcting hyperopia and presbyopia using a laser and an inlay outside the visual axis of eye

(76) Inventor: Gholam A. Peyman, New Orleans, LA (US)

Correspondence Address: BELL, BOYD, & LLOYD LLC, P. O. BOX 1135, CHICAGO, IL, 60690-1135, US

(21) Appl. No.: 10/917,492

(22) Filed: Aug. 13, 2004

(51) Int. Cl. A61F 9/008

(52) U.S. Cl. 606/5; 623/5.11; 128/898

(57) ABSTRACT

A cornea is reshaped by first creating a first cut in the cornea using an ultra-short pulse laser. The first cut is located below the surface of the cornea and does not extend through the epithelium. A second cut is then created using the ultra-short pulse laser. The second cut creates a corneal flap and intersects with the first cut to create a substantially severed portion of the cornea located between the first cut and the second cut. The severed portion of the cornea is located outside of the visual axis of the eye. The corneal flap is lifted away from the severed portion, and the severed portion is removed from the eye. The corneal flap is moved into the space on the cornea previously occupied by the severed portion. The cornea is thereby reshaped, and the reshaped portion of the cornea has an increased refractive power, correcting for hyperopic and presbyopic conditions.

### FIELD OF THE INVENTION

The present invention generally relates to the correction of refractive errors in an eye. More specifically, the present invention relates to a method of correcting refractive errors in an eye by reshaping the cornea to provide increased refractive power for near vision.

### BACKGROUND OF THE INVENTION

A normal emetropic eye includes a cornea, a lens, and a retina. The cornea and lens cooperatively focus light entering the eye from a far point—i.e. infinity—onto the retina. An eye can have a disorder known as ametropia, however. Ametropia is the inability of the lens and cornea to focus the far point correctly on the retina. Typical types of ametropia are myopia, hypermetropia or hyperopia, and astigmatism.

A hypermetropic or hyperopic eye has an axial length shorter than that of a normal emetropic eye, or a lens or cornea with a refractive power less than that of a lens and cornea of an emetropic eye. This lesser refractive power causes light entering the eye to be focused in back of the retina rather than onto the retina. Hyperopia typically affects close-up vision.

Another disorder that affects close-up vision is presbyopia. Presbyopia is an age related condition, and it is generally believed to be caused by a hardening of the lens of the eye that results in diminished ability to properly focus light entering the eye onto the retina.

A common method of treating a hyperopic or presbyopic eye is to place a “plus” or convex lens in front of the eye (i.e. glasses or contact lenses). Surgical techniques also exist for correcting a hyperopic or presbyopic eye. For example, U.S. Pat. No. 6,213,997 to Hood et al., which is hereby incorporated by reference in its entirety, discloses a method of reshaping a cornea of an eye to correct for hyperopia or presbyopia. The method disclosed in that patent is generally referred to as thermokeratoplasty and involves the application of thermal energy around the periphery of the surface of the cornea. The thermal treatment contracts the tissue, causing the central cornea to steepen. The steepened cornea has a higher refractive power, thereby improving the vision of the hyperopic or presbyopic eye. U.S. Pat. No. 5,779,676 to Kriesel et al., which is hereby incorporated by reference in its entirety, discloses a similar method which uses light energy to reshape the cornea.

Although these techniques have been used to successfully modify hyperopic and presbyopic eyes, a continuing need exists for improved methods for modifying a cornea to correct hyperopic and presbyopic conditions.

### SUMMARY OF THE INVENTION

An object of the present invention is to provide a method for modifying a cornea to correct hyperopic and presbyopic conditions.

Another object of the present invention is to provide a method for reshaping a cornea by using an ultra-short pulse laser.

A further object of the present invention is to provide a method for reshaping a cornea by removing a portion of the cornea.

A still further object of the present invention is to provide a method for resphaping a cornea by utilizing a corneal inlay.

Yet another object of the present invention is to provide a method for resphaping a cornea which provides for post-operative adjustments of the shape of the cornea using light.

The foregoing objects are basically obtained by a method of reshaping a cornea using an ultra-short pulse laser. The first step is to create a first cut in the cornea using the ultra-short pulse laser. The first cut is located below the surface of the cornea and does not extend through the epithelium. Next, a second cut is created in the cornea using the ultra-short pulse laser. The second cut creates a corneal flap and intersects with the first cut to create a substantially severed portion of the cornea located between the first cut and the second cut. The corneal flap is lifted away from the severed portion, and the severed portion is removed from the eye. The corneal flap is moved into the space on the cornea previously occupied by the severed portion. The cornea is thereby reshaped, and the reshaped portion of the cornea has an increased refractive power, correcting for hyperopic and presbyopic conditions.

If there are any residual refractive errors after performing the above-described steps, the cornea can be further reshaped by implanting a corneal inlay under the corneal flap. The corneal inlay is preferably formed of a light adjustable material so that the refractive power of the cornea can be adjusted after the corneal operation has been completed.

Other objects, advantages, and salient features of the present invention will become apparent from the following detailed description, which, taken in conjunction with the annexed drawings, discloses preferred embodiments of the invention.

### BRIEF DESCRIPTION OF THE DRAWINGS

Referring to the drawings which form a part of this disclosure:

FIG. 1A is a diagrammatic cross-sectional view of a portion of a cornea illustrating the creation of a first cut in the cornea;

FIG. 1B is a diagrammatic cross-sectional view of a portion of a cornea illustrating the creation of a second cut in the cornea of FIG. 1A;

FIG. 1C is a diagrammatic cross-sectional view of a portion of a cornea illustrating the lifting of a corneal flap created in FIG. 1B;

FIG. 1D is a diagrammatic cross-sectional view of a portion of a cornea illustrating the removal of a severed portion of the cornea created in FIG. 1C;

FIG. 1E is a diagrammatic cross-sectional view of a portion of a cornea with a restored corneal flap placed in the area of the removed severed portion as in FIG. 1D;

FIG. 1F is a diagrammatic cross-sectional view of a portion of a cornea with the flap of FIG. 1E raised and a corneal inlay implanted below the flap and in the area of the removed severed portion as shown in FIG. 1C;

FIG. 1G is a diagrammatic cross-sectional view of a portion of a cornea with an implanted corneal inlay of FIG. 1F and a restored corneal flap;

FIG. 1H is a diagrammatic cross-sectional view of a portion of a cornea of FIG. 1G with a corneal inlay that has been irradiated and expanded in its central portion;

FIG. 1I is a diagrammatic cross-sectional view of a portion of a cornea of FIG. 1G with a corneal inlay that has been irradiated and expanded on its peripheries;

FIG. 2 is a top plan view of a cornea with an implanted corneal inlay which extends 360 degrees around the cornea;

FIG. 3 is a top plan view of the corneal inlay illustrated in FIG. 2;

FIG. 4 is a top plan view of a cornea with an implanted corneal inlay which extends approximately 180 degrees around the cornea;

FIG. 5 is a top plan view of the corneal inlay illustrated in FIG. 4;

FIG. 6 is a top plan view of a cornea with a two part implanted corneal inlay;

FIG. 7 is a top plan view of a corneal inlay which extends through an arc of approximately 270 degrees;

FIG. 8 is a top plan view of a corneal inlay which extends through an arc of approximately 90 degrees;

FIG. 9 is a top plan view of a corneal inlay which extends through an arc of approximately 45 degrees;

FIG. 10A illustrates a second general embodiment of the invention and is a diagrammatic cross-sectional view of a portion of a cornea illustrating the creation of a first cut in the cornea leaving a hinge at the inner side of the cut;

FIG. 10B is a diagrammatic cross-sectional view of a portion of a cornea illustrating the lifting of the corneal flap of FIG. 10A;

FIG. 10C is a diagrammatic cross-sectional view of a portion of a cornea illustrating the creation of a second cut in the cornea of FIG. 10B;

FIG. 10D is a diagrammatic cross-sectional view of a portion of a cornea shown in FIG. 10C illustrating the removal of a severed portion of the cornea;

FIG. 11 illustrates a third general embodiment of the invention and is a diagrammatic cross-sectional view of a portion of a cornea illustrating a portion of the cornea that has been softened using an ultra-short pulse laser;

FIG. 12 illustrates a fourth general embodiment of the invention and is a diagrammatic cross-sectional view of a portion of a cornea illustrating the removal of a portion of the cornea using an electrical drill;

FIG. 13 is a side elevational view of a drill suitable for use with the method illustrated in FIG. 12;

FIG. 14 is a side elevational view in partial cross-section of another drill with a depth control shroud suitable for use with the method illustrated in FIG. 12; and

FIG. 15 illustrates a fifth general embodiment of the invention and is a diagrammatic cross-sectional view of a portion of a cornea illustrating a portion of the cornea which has been coagulated by applying thermal energy.

### DETAILED DESCRIPTION OF THE INVENTION

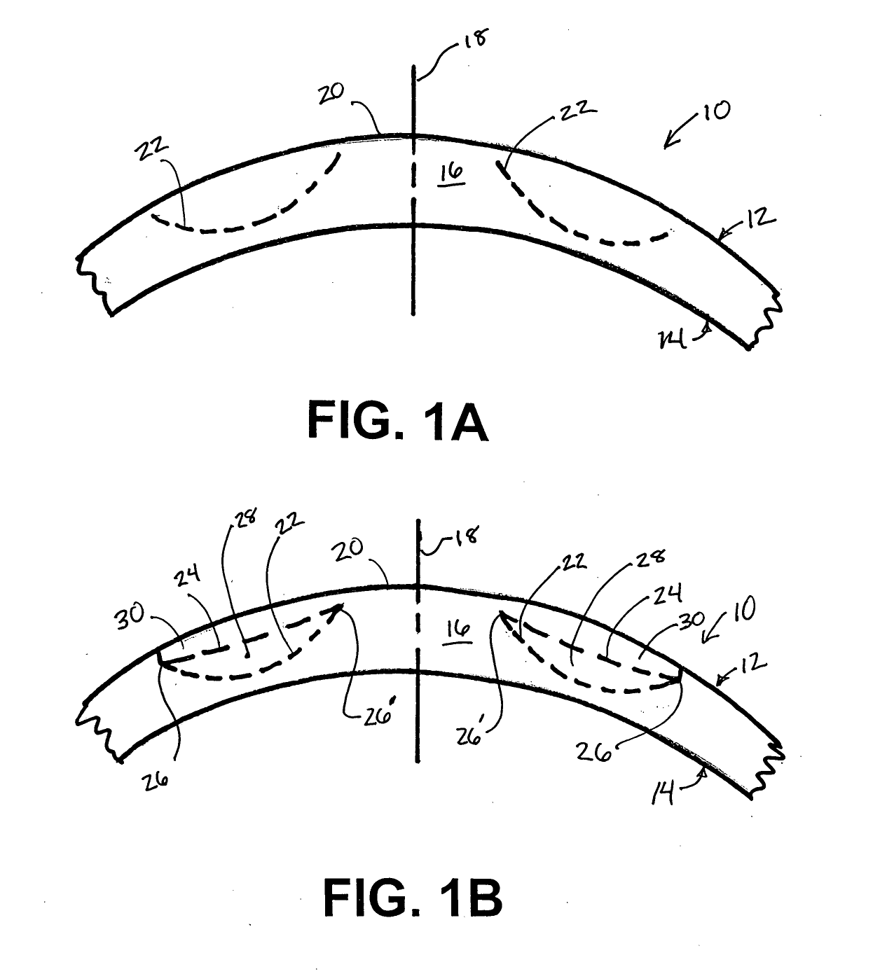

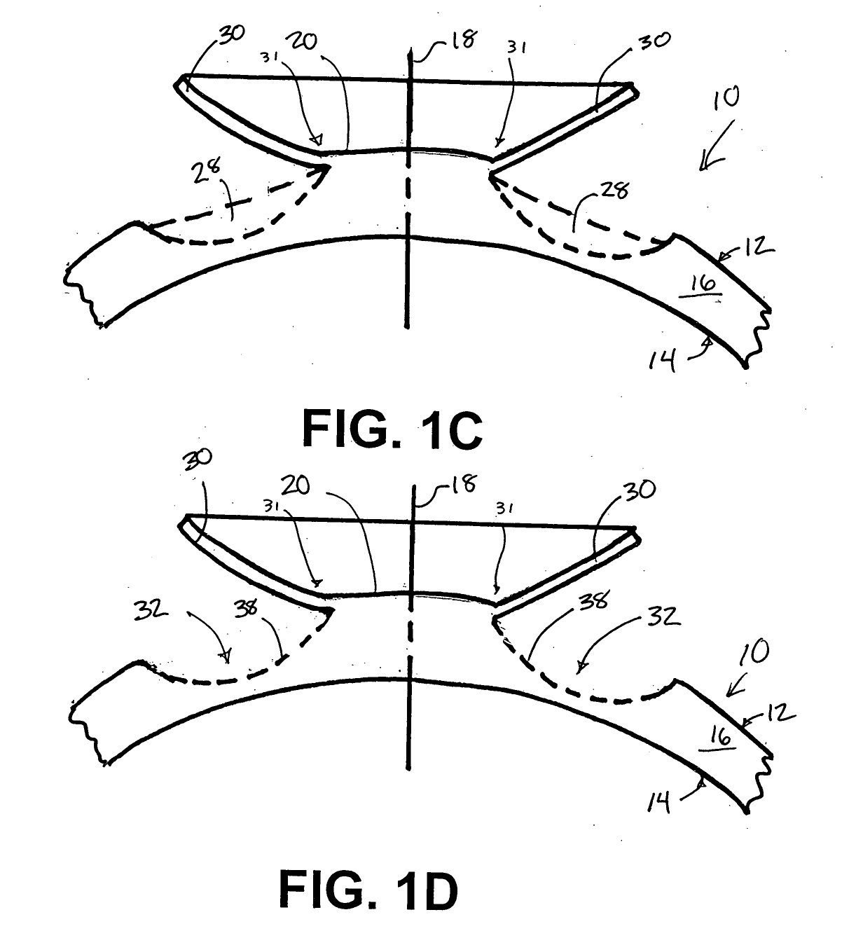

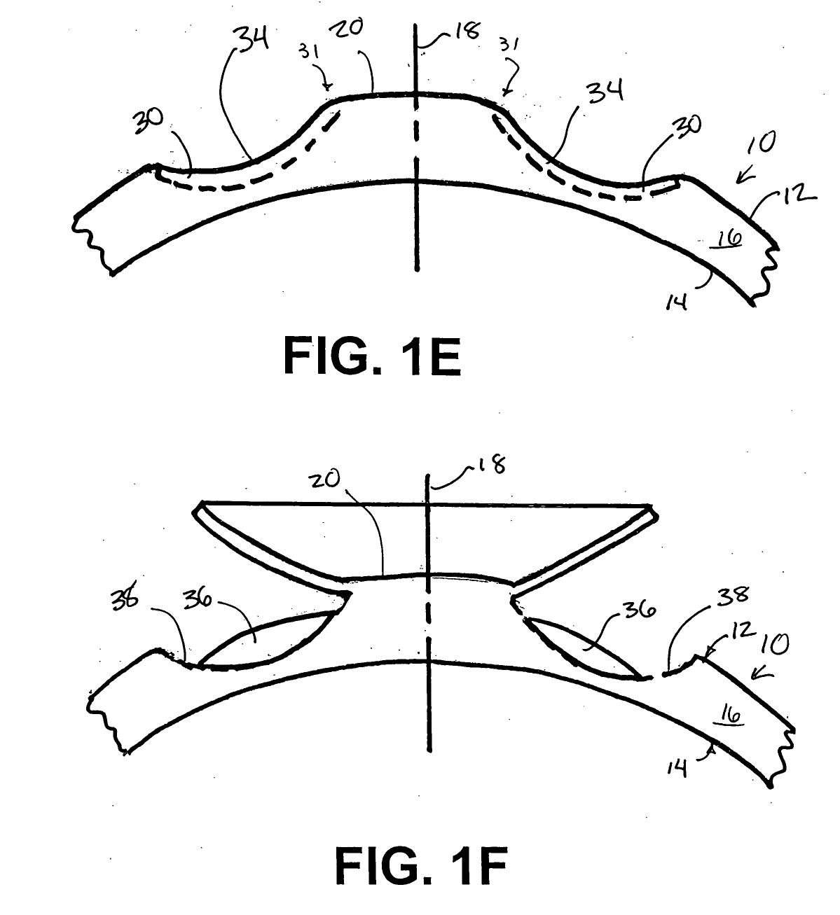

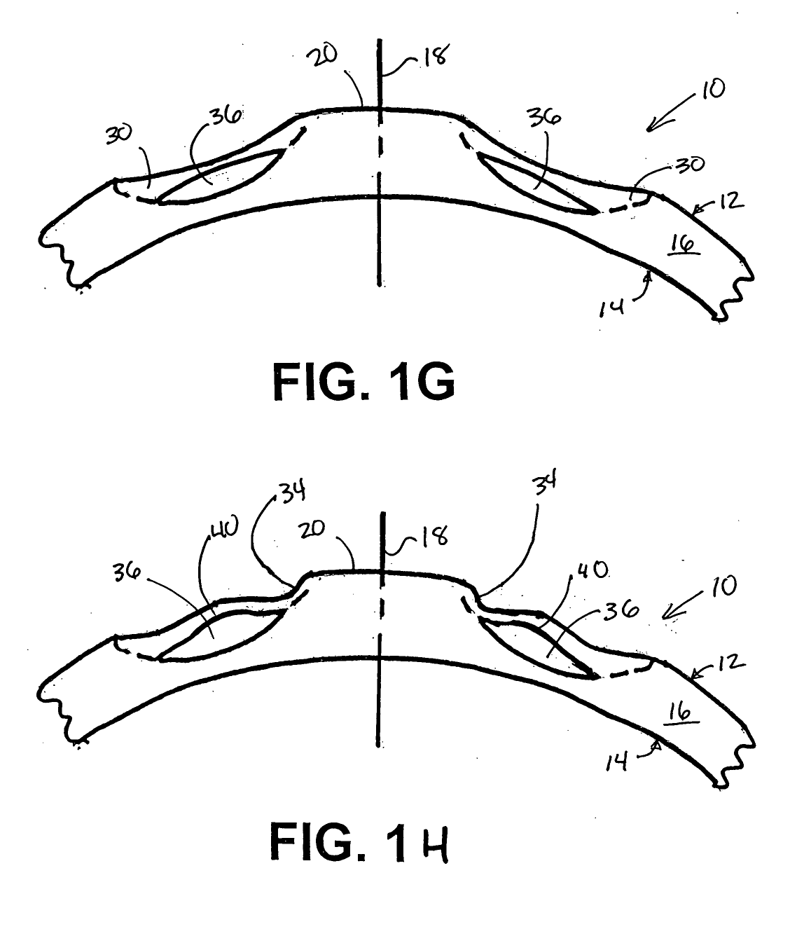

FIGS. 1A-1I illustrate a method of correcting hyperopic or presbyopic errors in an eye according to a first embodiment of the present invention. As seen in those figures, the cornea 10 has an epithelium 12 (the external, exposed layer of the cornea), an endothelium 14 (the internal layer of the cornea), and a stroma 16 located therebetween. The cornea 10 has a central visual or optical axis 18 which passes through the central area 20 of the cornea 10.

To correct for hyperopia or presbyopia, the refractive power of the cornea 10 must be increased. In the method of the present invention, this is accomplished by reshaping the cornea 10. As seen in FIG. 1A, a first curvilinear cut 22 is created in the stroma 16 using an ultra-short pulse laser, such as a femtosecond laser. The use of an ultra-short pulse laser is desirable because such a laser creates precise intrastromal cuts with little corneal trauma. A suitable ultra-short pulse laser is the IntraLase FS laser available from the IntraLase Corporation based in Irvine, California. The cut is made outside the visual axis 18, and as described below, can encompass any required angular extent, such as, for example, 45, 90, 180, 270 or 360 degrees. Preferably the angular cut has its radius of curvature aligned with visual axis 18.

After creating the first cut 22, a second cut 24 is made through the stroma 16 using the ultra-short pulse laser and it has an angular extent substantially the same as that of the first cut. As seen in FIG. 1B, the second cut 24 is located closer to the epithelium 12 than the first cut 22. The second cut 24 intersects the first cut 22 at two opposite intersection areas 26 and 26′ to form a severed portion 28 of the cornea between the two cuts. The second cut 24 also extends through the epithelium 12 above area 26 to create a corneal flap 30. After creating the second cut 24, as seen in FIG. 1C, the cornea flap 30 is lifted away about hinge 31 from the severed portion 28 of the cornea to provide access to the severed portion 28 of the cornea 10. The severed portion 28 of the cornea 10 is then removed, as shown in FIG. 1D, leaving behind a space 32 that was previously occupied by the severed portion 28 of the cornea 10. After removing the severed portion 28, the corneal flap 30 is closed by moving the corneal flap 30 about hinge 31 into the space 32 previously occupied by the severed portion of the cornea. Thus, as seen in FIG. 1E, after the corneal flap 30 is replaced, the cornea has a bifocal nature. The central area 20, which has not been disturbed and has its original thickness, provides undisturbed far vision. The adjacent, reshaped portion 34 of the cornea 10 has a reduced thickness and thus an increased refractive power, providing corrected near vision.

The volume, i.e. the thickness, of the severed portion 28 can be varied according to the desired amount of correction. It is expected that a severed portion approximately 25 to 35 microns thick will be sufficient to correct presbyopia of 3 diopters. The flap 30 preferably has a radial thickness or width of about 1 millimeter to about 9 millimeters. Further, the angular extent of the corneal flap can vary depending on the nature of the desired treatment. To treat a hyperopic eye, the corneal flap should extend 360 degrees around the visual axis of the eye, while for a presbyopic eye, the corneal flap should extend between approximately 150 to 250 degrees around the visual axis of the eye.

After performing the above method steps, the eye is allowed to at least partially heal so that the vision becomes stabilized and any significant swelling caused by the operation reduces. Once a suitable period of time has elapsed, the eye is measured to determine if there are any remaining refractive errors which require further correction. If there are any remaining errors, the corneal flap 30 is lifted, and a corneal inlay 36 is implanted into the cornea 10 on the exposed stroma 38, as shown in the FIG. 1F. The corneal flap 30 is once again closed and the eye is again allowed to heal.

The corneal inlay 36 can be made of any suitable bio-compatible material, but preferably it is made of a light adjustable material and has a refractive power the same or different than that of the cornea. The use of a light adjustable material provides another opportunity to adjust the amount of refractive power in the post-operative period. If there are any residual refractive errors, portions of the corneal inlay 36 may be irradiated to alter the refractive properties of the cornea. FIG. 1H illustrates an example of irradiating the central portion 40 of the cornea inlay 36 of FIG. 1G to increase the refractive power of the inlay 36 while also increasing the central steepness of the reshaped portion 34 of the cornea 10. The net refractive correction is the degree of dioptic power increase in the inlay 36 minus the degree of reduction of the refractive power of the cornea in the reshaped area, the sum of which should not be zero. If the sum of this correction is 1, 2, 3, or more plus dioptic power, the result is correction of presbyopia and hyperopia. FIG. 1I illustrates an example of irradiating the outer periphery 42 and inner periphery 44 of the corneal inlay 36 to decrease the refractive power of the inlay 36 while decreasing the central steepness of the reshaped portion 34 of the cornea 10. U.S. Pat. No. 6,749,632 to Sandstedt et al., which is hereby incorporated by reference in its entirety, discloses suitable light adjustable materials and methods of using those materials in optical implants.

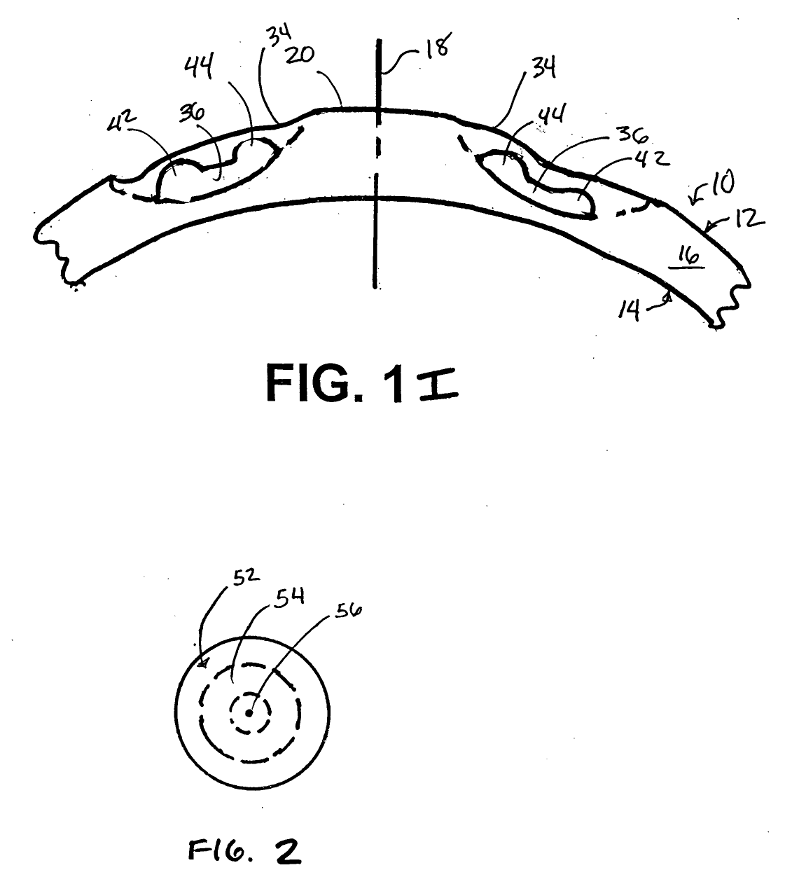

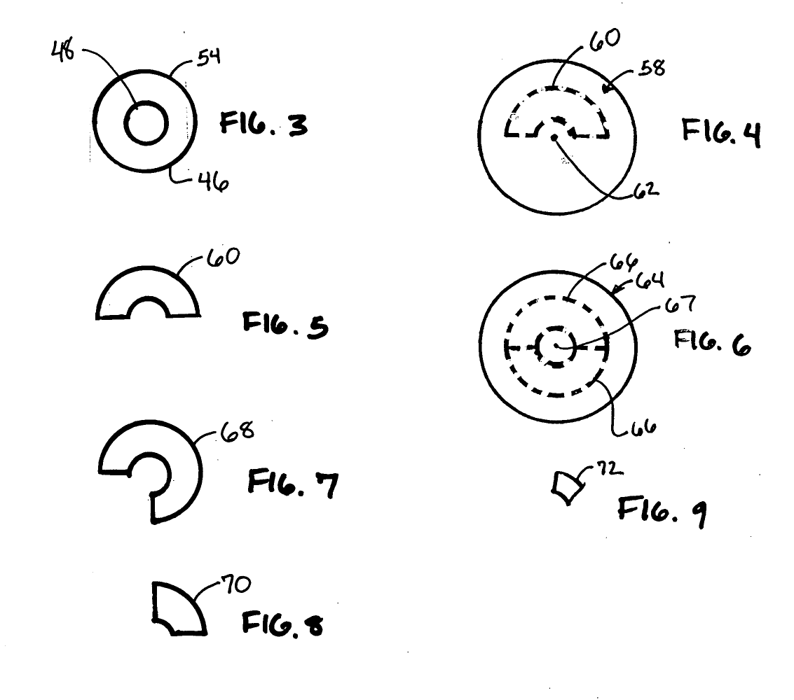

FIG. 2 illustrates a top view of a cornea 52 with a corneal inlay 54 and FIG. 3 illustrates the corneal inlay 54 prior to implantation. The corneal inlay 54 of those figures is designed to correct for hyperopic conditions and comprises a 360 degree ring that extends substantially all around the visual axis 56 of the eye. The corneal inlay may optionally be tinted around the outer edge 46 and the inner edge 48, preferably by black paint or the like. The tinting helps to minimize visual artifacts produced by the dispersion of light at the edges of the inlay 54 that is caused by the different indices of refraction of the inlay and the cornea.

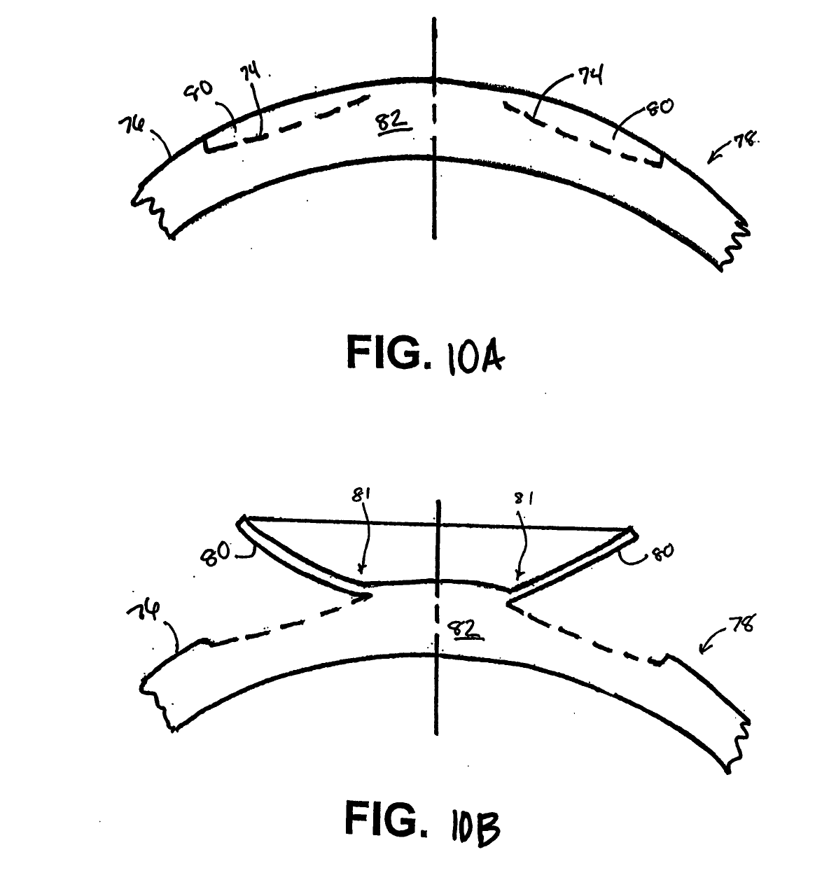

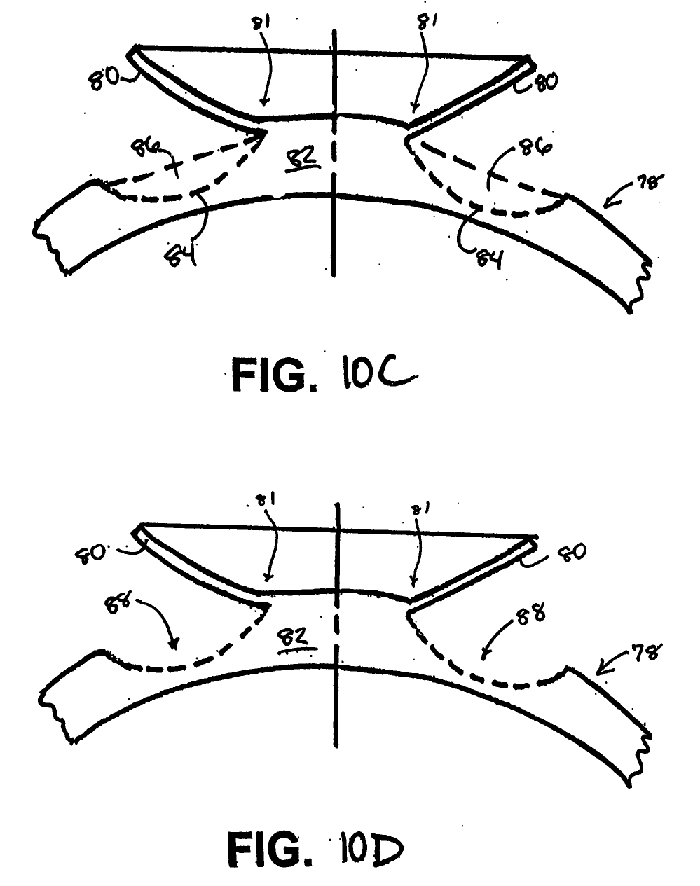

For treating presbyopic conditions, the inlay preferably does not extend complete around the visual axis of the eye. FIG. 4 illustrates a cornea 58 with an inlay 60 which extends substantially 180 degrees around the visual axis 62 of the eye, and FIG. 5 illustrates the inlay 60 prior to implantation. FIG. 6 illustrates a cornea 64 with two 180 degree inlays 66 implanted to form an inlay which extends completely around the visual axis 67 of the eye. The use of two inlays 66 allows easier implantation as compared to a single 360 degree inlay. FIGS. 7, 8, and 9 illustrate further inlays that are suitable for use in the present invention. FIG. 7 illustrates an inlay 68 that extends substantially 270 degrees around the visual axis of the eye, FIG. 8 illustrates an inlay 70 that extends substantially 90 degrees around the visual axis of the eye, and FIG. 9 illustrates an inlay 72 that extends substantially 45 degrees around the visual axis of the eye. Of course, all of the illustrated inlays are only exemplary in nature and the choice of the particular size, shape, and number of inlays will be dictated by the particular characteristics of the eye under treatment.

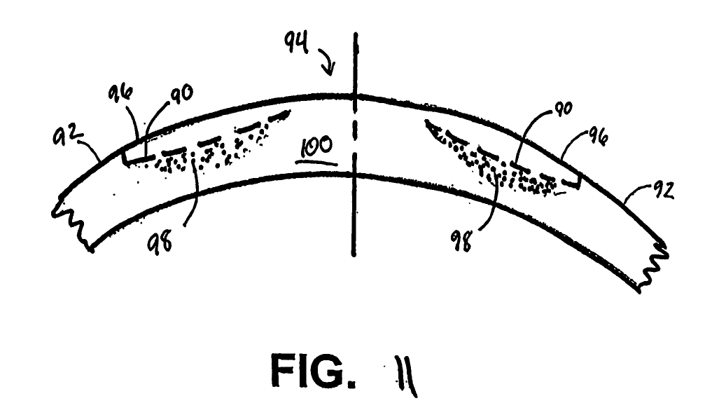

### Embodiment of FIGS. 10A-10D

FIGS. 10A-10D illustrate a second embodiment of the present invention. This second embodiment is similar to the first embodiment of FIGS. 1-9 except that the flap is made by the first cut and the portion to be severed and removed is made by the second cut. In this second embodiment, an ultra-short pulse laser makes a first cut 74 through the epithelium 76 of the cornea 78 to create a corneal flap 80, as seen in FIG. 10A. The corneal flap 80 is lifted about hinge 81 to expose a portion of the stroma 82, as illustrated in FIG. 10B. Next, as seen in FIG. 10C, an ultra-short pulse laser is used to make a second cut 84 through the stroma 82 and create a severed portion 86 of the cornea 78 between the two cuts. The severed portion 86 of the cornea 78 is then removed, as shown in FIG. 10D, leaving behind a space 88 that was previously occupied by the severed portion 86 of the cornea 78. The remainder of the treatment takes place as discussed above with respect to the first embodiment.

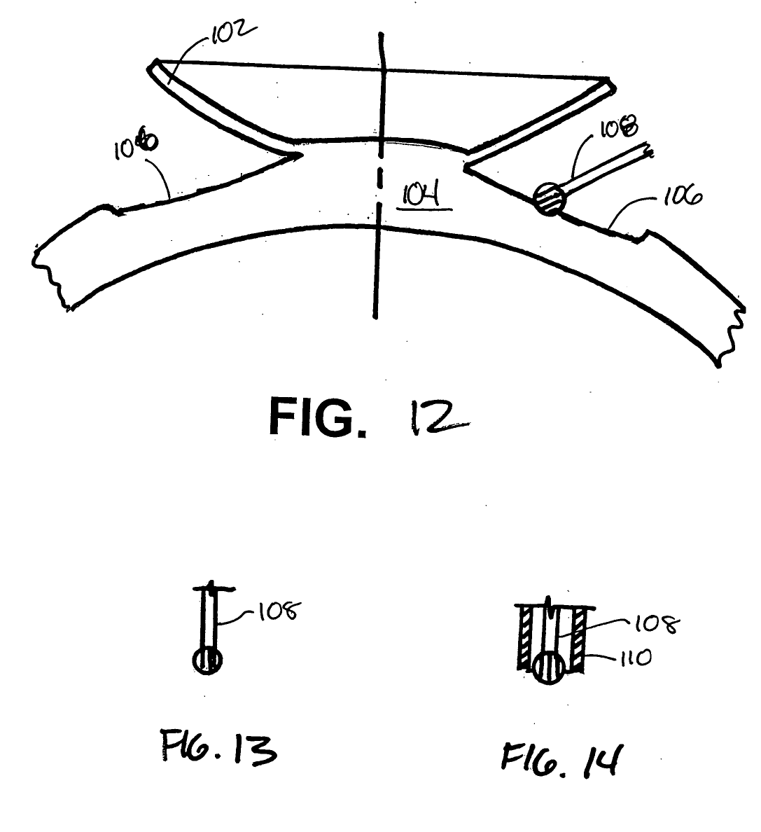

### Embodiment of FIG. 11

FIG. 11 illustrates a third embodiment of the present invention. In this embodiment, an ultra-short pulse laser makes a first cut 90 through the epithelium 92 of the cornea 94 to create a corneal flap 96. The laser is then used to soften a portion 98 of the cornea by selectively aiming and firing the laser at a portion of the stroma 100 located below the corneal flap 96. The corneal flap 96 can then be lifted, and the softened portion 98 of the stroma 100 can be removed using a spatula or any other suitable instrument. After the softened portion has been removed, the remainder of the treatment takes place as discussed above with respect to the first embodiment.

### Embodiment of FIGS. 12-14

FIGS. 12-14 illustrate a fourth embodiment of the present invention. In this embodiment, a corneal flap 102 is formed and raised to form an exposed portion 106 of the stroma 104. A drill bit 108 is then used to remove a portion of the stroma 104. Once the desired amount of stroma 104 has been removed, the remainder of the treatment takes place as discussed above with respect to the first embodiment. The drill bit 108 used to perform the drilling operation can be unshrouded, as shown in FIG. 13, or the drill bit 108 can have a shroud 110, as shown in FIG. 14. The shroud provides precise depth control.

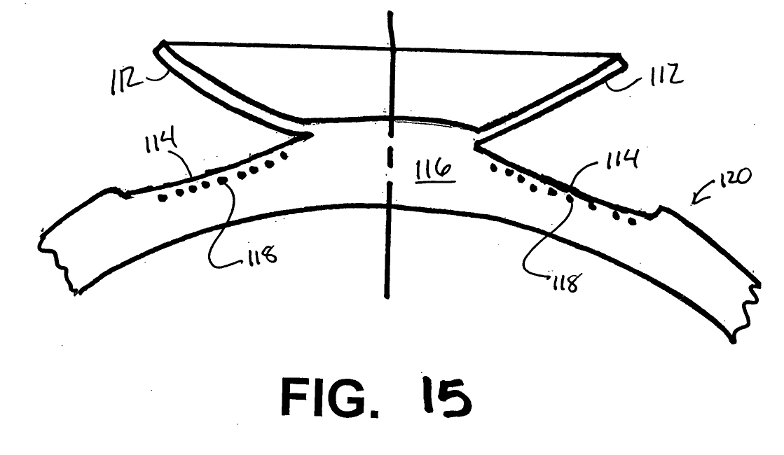

### Embodiment of FIG. 15

FIG. 15 illustrates a fifth embodiment of the present invention. In this embodiment, a corneal flap 112 is formed and raised as previously discussed to form an exposed portion 114 of the stroma 116. Heat is applied to the exposed portion 114 of the stroma 116. The heat is sufficient to cause the heated portion 118 of the stroma 116 to shrink, thereby reshaping the cornea 120 by increasing the steepness of the cornea 120. The heat may be high enough to cause the heated portion to coagulate. The corneal flap 112 is then closed and allowed to at least partially heal. The remainder of the treatment takes place as discussed above with respect to the first embodiment.

While various embodiments have been chosen to illustrate the invention, it will be understood by those skilled in the art that various changes and modifications can be made therein without departing from the scope of the invention as defined in the appended claims.

What is claimed is:

1. A method of correcting refractive errors in an eye, comprising the steps of:
creating a first cut in a cornea of the eye with an ultra-short pulse laser, the first cut being located below the surface of the cornea;
creating a second cut in the cornea using the ultra-short pulse laser, the second cut creating a corneal flap, the second cut creating a substantially severed portion of the cornea located between the first cut and the second cut, the severed portion of the cornea being located outside of the visual axis of the eye;
lifting the corneal flap away from the severed portion;
removing the severed portion of the cornea from the cornea; and
closing the corneal flap by moving the corneal flap into the space on the cornea previously occupied by the severed portion.

2. A method according to claim 1, further comprising the steps of:
allowing the cornea to partially heal after closing the corneal flap;
measuring the eye to determine if there are any refractive errors necessitating correction after the eye has partially healed;
lifting the corneal flap away from the cornea;
implanting a corneal inlay to correct any remaining refractive errors;
and closing the corneal flap onto the corneal inlay by moving the corneal flap.

3. A method according to claim 2, wherein
the corneal inlay extends substantially all around the visual axis of the eye through 360 degrees.

4. A method according to claim 2, wherein
the corneal inlay extends between substantially 180 and substantially 270 degrees around the visual axis of the eye.

5. A method according to claim 2, wherein
the corneal inlay extends between substantially 90 and substantially 180 degrees around the visual axis of the eye.

6. A method according to claim 2, wherein
the corneal inlay extends between substantially 45 and substantially 90 degrees around the visual axis of the eye.

7. A method according to claim 2, further comprising the step of:
irradiating the corneal inlay to correct visual errors in the eye.

8. A method according to claim 2, wherein
the inner and outer edges of the corneal inlay are tinted.

9. A method of correcting refractive errors in an eye, comprising the steps of:
creating a first cut in the cornea using an ultra-short pulse laser; the first cut creating a corneal flap;
lifting the corneal flap to expose a portion of the stroma;
creating a second cut in the cornea using the ultra-short pulse laser, the second cut creating a substantially severed portion of the cornea located between the first cut and the second cut, the severed portion of the cornea being located outside of the visual axis of the eye;
removing the severed portion of the cornea from the cornea; and
closing the corneal flap by moving the corneal flap into the space on the cornea previously occupied by the severed portion.

10. A method according to claim 9, further comprising the steps of:
allowing the cornea to partially heal after closing the corneal flap;
measuring the eye to determine if there are any refractive errors necessitating correction after the eye has partially healed;
lifting the corneal flap away from the cornea;
implanting a corneal inlay to correct any remaining refractive errors;
and closing the corneal flap by moving the corneal flap onto the corneal inlay.

11. A method according to claim 10, wherein
the corneal inlay extends between substantially all around the visual axis of the eye through 360 degrees.

12. A method according to claim 10, wherein
the corneal inlay extends between substantially 180 and substantially 270 degrees around the visual axis of the eye.

13. A method according to claim 10, wherein
the corneal inlay extends between substantially 90 and substantially 180 degrees around the visual axis of the eye.

14. A method according to claim 10, wherein
the corneal inlay extends between substantially 45 and substantially 90 degrees around the visual axis of the eye.

15. A method according to claim 10, further comprising the step of:
irradiating the corneal inlay to correct visual errors in the eye.

16. A method according to claim 10, wherein
the inner and outer edges of the corneal inlay are tinted.

17. A method of correcting refractive errors in an eye, comprising the steps of:
firing an ultra-short pulse laser at a cornea to soften tissue in the cornea, the softened tissue being located outside of the visual axis of the eye;
creating a corneal flap using the ultra-short pulse laser;
lifting the corneal flap to expose the softened tissue of the cornea;
removing the softened tissue; and
restoring the corneal flap.

18. A method according to claim 17, further comprising the steps of:
allowing the eye to partially heal after restoring the corneal flap;
measuring the eye to determine if there are any refractive errors after the eye has partially healed; and
implanting a corneal inlay to correct any remaining refractive errors.

19. A method according to claim 18, wherein the corneal inlay extends between substantially all around the visual axis of the eye through 360 degrees.

20. A method according to claim 18, wherein
the corneal inlay extends between substantially 180 and substantially 270 degrees around the visual axis of the eye.

21. A method according to claim 18, wherein
the corneal inlay extends between substantially 90 and substantially 180 degrees around the visual axis of the eye.

22. A method according to claim 18, wherein
the corneal inlay extends between substantially 45 and substantially 90 degrees around the visual axis of the eye.

23. A method according to claim 18, further comprising the step of:
irradiating the corneal inlay to correct visual errors in the eye.

24. A method according to claim 18, wherein
the inner and outer edges of the corneal inlay are tinted.

25. A method according to claim 17, wherein
the softened tissue is removed using a spatula.

26. A method according to claim 18, wherein
the softened tissue is removed using a drill.

27. A method according to claim 18, wherein
the softened tissue is removed using a drill with a shroud.

28. A method of correcting refractive errors in an eye, comprising the steps of:
creating a corneal flap using an ultra-short pulse laser;
lifting the corneal flap to expose a portion of the stroma;
removing a portion of the stroma using a drill, the removed portion of the stroma being located outside of the visual axis of the eye; and
restoring the corneal flap.

29. A method according to claim 28, further comprising the steps of:
allowing the cornea to partially heal after closing the corneal flap;
measuring the eye to determine if there are any refractive errors necessitating correction after the eye has partially healed;
lifting the corneal flap away from the cornea;
implanting a corneal inlay to correct any remaining refractive errors;
and closing the corneal flap by moving the corneal flap onto the corneal inlay.

30. A method according to claim 29, wherein
the corneal inlay extends substantially all around the visual axis of the eye through 360 degrees.

31. A method according to claim 29, wherein
the corneal inlay extends between substantially 180 and substantially 270 degrees around the visual axis of the eye.

32. A method according to claim 29, wherein
the corneal inlay extends between substantially 90 and substantially 180 degrees around the visual axis of the eye.

33. A method according to claim 29, wherein
the corneal inlay extends between substantially 45 and substantially 90 degrees around the visual axis of the eye.

34. A method according to claim 29, further comprising the step of:
irradiating the corneal inlay to correct visual errors in the eye.

35. A method according to claim 29, wherein
the inner and outer edges of the corneal inlay are tinted.

36. A method of correcting refractive errors in an eye, comprising the steps of:
creating a corneal flap using an ultra-short pulse laser;
lifting the corneal flap to expose a portion of the stroma;
treating a portion of the exposed stroma to cause it to shrink, the exposed portion of the stroma being located outside of the visual axis of the eye; and
restoring the corneal flap.

37. A method according to claim 36, wherein
the treating step comprises heating.

38. A method according to claim 36, wherein
the treating step comprises coagulating.

39. A method according to claim 36, further comprising the steps of:
allowing the cornea to partially heal after closing the corneal flap;
measuring the eye to determine if there are any refractive errors necessitating correction after the eye has partially healed;
lifting the corneal flap away from the cornea;
implanting a corneal inlay to correct any remaining refractive errors;
and closing the corneal flap by moving the corneal flap onto the corneal inlay.

40. A method according to claim 39, wherein
the corneal inlay extends substantially all around the visual axis of the eye through 360 degrees.

41. A method according to claim 39, wherein
the corneal inlay extends between substantially 180 and substantially 270 degrees around the visual axis of the eye.

42. A method according to claim 39, wherein
the corneal inlay extends between substantially 90 and substantially 180 degrees around the visual axis of the eye.

43. A method according to claim 39, wherein
the corneal inlay extends between substantially 45 and substantially 90 degrees around the visual axis of the eye.

44. A method according to claim 39, further comprising the step of:
irradiating the corneal inlay to correct visual errors in the eye.

45. A method according to claim 39, wherein
the inner and outer edges of the corneal inlay are tinted.
